(19) United States
(12) Patent Application Publication
(10) Pub. No.: US 2017/0295737 A1
(43) Pub. Date: Oct. 19, 2017

(54) Cotton variety DP 348 RF PIMA

(71) Applicants: James Michael Olvey, Maricopa, AZ (US); James Michael Olvey, II, Maricopa, AZ (US)

(72) Inventors: James Michael Olvey, Maricopa, AZ (US); James Michael Olvey, II, Maricopa, AZ (US)

(21) Appl. No.: 14/999,261

(22) Filed: Apr. 15, 2016

(51) Int. Cl. A01H 5/08; C12N 15/82; A01H 1/02

(52) U.S. Cl. CPC A01H 5/08; C12N 15/8289; C12N 15/8279; C12N 15/8278; C12N 15/8275; A01H 1/02; C12N 15/8271; C12N 15/8247; C12N 15/8245; C12N 15/8241; C12N 15/8274

(57) ABSTRACT

The invention relates to the novel cotton variety designated DP 348 RF PIMA. Provided by the invention are the seeds, plants, plant parts and derivatives of the cotton variety DP 348 RF PIMA. Also provided by the invention are methods of using cotton variety DP 348 RF PIMA and products derived therefrom. Still further provided by the invention are methods for producing cotton plants by crossing the cotton variety DP 348 RF PIMA with itself or another cotton variety and plants and seeds produced by such methods.

### BACKGROUND OF THE INVENTION

### 1. Field of the Invention

The present invention relates generally to the field of plant breeding. In particular, the invention relates to a novel cotton cultivar DP 348 RF PIMA.

### 2. Background of the invention

The two cotton species commercially grown in the United States are Gossypium hirsutum, commonly known as short staple or upland cotton and Gossypium barbadense, commonly known as extra long staple (ELS) or, in the United States, as Pima cotton. Upland cotton fiber is used in a wide array of coarser spin count products. Pima cotton is used in finer spin count yarns (50-80) which are primarily used in more expensive garments. Other properties of Pima cotton are critical because of fiber end use.

Cotton is an important and valuable field crop. Thus, a continuing goal of plant breeders is to develop stable, high yielding cotton varieties that are agronomically sound. The reasons for this goal are obviously to maximize the amount and quality of the fiber produced on the land used and to supply fiber, oil and food for animals and humans. To accomplish this goal, the cotton breeder must select and develop plants that have the traits that result in superior cultivars.

Important traits for cotton include higher fiber (lint) yield, earlier maturity, improved fiber quality, resistance to diseases and insects, tolerance to drought and heat, and improved agronomic traits. A breeder initially selects and crosses two or more parental lines, followed by generation advancement and selection, thus producing many new genetic combinations. The breeder can theoretically generate extremely large number of different genetic combinations via this procedure.

### SUMMARY OF THE INVENTION

The invention relates to seeds, plants, plant cells, parts of plants, cotton lint or fiber, and cotton textiles of cotton variety DP 348 RF PIMA as well as to hybrid cotton plants and seeds obtained by repeatedly crossing plants of DP 348 RF PIMA with other cotton plants, and Essentially Derived Varieties of cotton variety DP 348 RF PIMA. The invention encompasses plants and plant varieties produced by the method of derivation or essential derivation from plants of DP 348 RF PIMA and to plants of DP 348 RF PIMA reproduced by vegetative methods, including but not limited to regeneration of embryogenic cells or tissue of DP 348 RF PIMA. The invention also encompasses methods of producing cotton seeds that comprise crossing plants of cotton variety DP 348 RF PIMA either with itself or with a second, distinct cotton plant.

### DETAILED DESCRIPTION OF THE INVENTION

The invention has been obtained by a general breed-ing process comprising the steps outlined below. For refeence, see chapter 11, “Breeding Self-Pollinated Crops by Hybridization and Pedigree Selection” in Briggs and Knowles (1967).

The invention provides, in one aspect, methods and composition relating to plants, seeds and derivatives of the cotton variety DP 348 RF PIMA. Cotton variety DP 348 RF PIMA was developed from an initial cross of P1/P2. The breeding history of the variety can be summarized as follows:

| Generation | Year | Description |
|---|---|---|
| Introgression | 2000-2008 | Monsanto's and Deltapine's Transgenic Introgression Program |
| | | for Pima's S-6 (P1) and S-7 (P1) into OA germplasm including |
| | | OA-303, 304, 305, 312, 340 and 357(P2) with Bollard 2 and |
| | | Roundup Ready Flex genes. |
| | | Cross was made between donor parent P1 (containing the gene |
| | | events B2 and RF) and conventional recurrent parent P2. |
| | | F1 Plants were grown in and advanced using bulk based on event of |
| | | interest selection. |
| | | BC1 Cross was made in Leland, MS using an F1 plant selection |
| | | and P2 as the recurrent parent. |
| | | BC1F1 Plants were grown in Leland, MS and advanced using |
| | | plant selection for event of interest. |
| | | BC2 Cross was made in Leland, MS using a BC1F1 plant |
| | | selection and P2 as the recurrent parent. |
| | | BC2F1 Plants were grown in Leland, MS and advanced using |
| | | plant selection based on event of interest and MABC recovery of |
| | | the recurrent parent. |
| | | BC3 Cross was made in Juana Diaz, PR using a BC2F1 plant |
| | | selection and P2 as the recurrent parent. |
| | | BC3F1 Plants were grown in Juana Diaz, PR and advanced using |
| | | plant selection based on event of interest selection and MABC |
| | | recovery of the recurrent parent. |
| | | BC4 Cross using a BC3F1 plant selection and P2 (containg the |
| | | gene event 88913 and for glyphosate and bt tolerance) as the |
| | | recurrent parent. |
| | | BC4F1 Plants were grown in Lubbock, TX and advanced using |
| | | plant selection based on events of interest selection and MABC |
| | | recovery of the recurrent parent. |
| | | BC5 Cross using a BC4F1 plant selection and P2 as the recurrent |
| | | parent. |
| | | F1 Plants were grown and advanced using plant selection based |
| | | on event of interest selection and recovery of the recurrent parent. |
| | | F2 Plants were grown and advanced using plant selection based |
| | | on homozygosity foe the intended events. |
| | | F3 Plants were grown and advanced using bulk based on gene |
| | | purity. |
| BC5F4 | 2008 | Made Individual Plant Selections in Deltapine/Monsanto |
| | | nursery at Bakerfield, CA in plot 8B2RF. |
| F5 | 2009 | O&A Enterprises made a Single Plant Selections at |
| | | O&A main nursery in Riverdale. CA. |
| F6 | 2010 | Evaluated in O&A nursery in California in Progeny rows. |
| F7 | 2011 | O&A placed into Fusarium Race 4 nursery in CA, made single |
| | | selections. |
| F8 | 2012 | Re-evaluated FOV4 single plant selection in Progeny rows. |
| F9 | 2013 | Advanced Strains Testing, State testing and small seed production. |
| | | Selected based on the lint yield, lint percent, fiber quality, maturity. |
| | | disease resistance to FOV-4 and Verticillium Wilt, transgene purity |
| | | and trueness to type with the recurrent parent. |
| F10 | 2014 | Advanced Strains Testing, State Testing and increased seed |
| | | production. |
| | | Selection based on the lint yield, lint percent, fiber quality, maturity, |
| | | disease resistance to FOV-4 and Verticillium Wilt, transgene purity, |
| | | and trueness to type with the recurrent parent. |
| F11 | 2015 | Advanced Strains Testing, State testing, increased seed production. |
| | | Selection based on the lint yield, lint percent, fiber quality, maturity, |
| | | disease resistance to FOV-4 and Verticillium Wilt, transgene purity, |
| | | and trueness to type with the recurrent parent. |

The cotton variety DP 348 RF PIMA has been judged to be uniform for breeding purposes and testing. The variety can be reproduced by planting and growing seeds of the variety under self-pollinating or sib-pollinating conditions, as is known to those of skill in the agricultural arts. Variety DP 348 RF PIMA shows no variants other than what would normally be expected due to environment or that would occur for almost any characteristic during the course of repeated sexual reproduction. The results of an objective description of the variety are presented below, in Table 1. Those of skill in the art will recognize that these are typical values that may vary due to environment and that other values that are substantially equivalent are within the scope of the invention.

TABLE 1
Phenotypic Description of Variety DP 348 RF PIMA
Species: Gossypium barbadense L

Areas of Adaptation:
Eastern NO
Delta NO
Central NO
Plains NO
Blacklands NO
Western YES
Arizona YES
California YES
New Mexico YES
West Texas YES
General:
Plant Habit: Intermediate
Foliage: Intermediate
Stem Lodging: Erect
Fruiting Branch: Normal
Growth: Indeterminate
Leaf Color: Dark Green

These are typical values. Values may vary due to environment. Other values that are substantially equivalent are within the scope of the invention.

Cotton variety DP 348 RF PIMA is an herbicide-tolerant cotton variety, as a result of containing events 88913. Event MON 88913 confers glyphosate tolerance by producing a 5-enolpyruvylshikimate-3-phosphate synthase protein from Agrobacterium sp. strain CP4 and is the subject of U.S. Pat. No. 7,381,861, the disclosure of which is incorporated herein by reference. Event MON 88913 is also covered by one or more of the following U.S. Pat. Nos.: 8,071,735; 7,141,722; 7,112,725; 6,949,696; 6,660,911; 6,083,878; 6,051,753; 5,728,925; 5,717,084.

The performance characteristics of cotton variety DP 348 RF PIMA were also analyzed and comparisons were made with competing varieties. The results of the analysis are presented in Table 1 below, DP 348 RF PIMA is compared to cotton varieties PHY 805 and PHY 811, the top 2 commercial pima varieties sold to farmers. The asterisk (*) indicates a significant difference from DP 348 RF PIMA at the 5% (LSD .05) confidence level.

Table 1: Yield Trait DP 348 RF PIMA Phy 805 Phy 811 LSD .05 CV% 2014-15 Lint Yield (lbs/ac) Corcoran 2094 1958* 1857* 84 2.8 Los Banos 1097 890* 1036 138 4.8 Westside 2007 1892 1912 137 5.9 Buttonwillow 2013 1758* 1932 254 12.3 Deputy TX/NM 1187 929* 910* 102 6.0 Los Banos 2 1047 936 926 206 13.1 Bakersfield 1553 1476 1459 196 4.0 Maricopa 1481 1222* 1173* 130 6.4 Mean 1560 1383* 1400* 140 8.0

3. Definitions

In the description and tables herein, a number of terms are used. In order to provide a clear and consistent understanding of the specification and claims, the following definitions are provided:

A: When used in conjunction with the word “comprising” or other open language in the claims, the words “a” and “an” denote “one or more.”

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Crossing: The mating of two parent plants.

Cross-pollination: Fertilization by the union of two gametes from different plants.

Desired Agronomic Characteristics: Agronomic characteristics (which will vary from crop to crop and plant to plant) such as yield, maturity, pest resistance and lint percent which are desired in a commercially acceptable crop or plant. For example, improved agronomic characteristics for cotton include yield, maturity, fiber content and fiber qualities.

Diploid: A cell or organism having two sets of chromosomes.

Disease Resistance: The ability of plants to restrict the activities of a specified pest, such as an insect, fungus, virus, or bacteria.

Disease Tolerance: The ability of plants to endure a specified pest (such as an insect, fungus, virus or bacteria) or an adverse environmental condition and still perform and produce in spite of this disorder.

Donor Parent: The parent of a variety which contains the gene or trait of interest which is desired to be introduced into a second variety.

ELS: The abbreviation for “Extra Long Staple.” ELS is the group classification for cotton in the longest staple length category. As used in practice and for commerce, ELS denotes varieties belonging to the species G. barbadense that have superior fiber qualities, including classification in the longest staple length category.

Emasculate: The removal of plant male sex organs or the inactivation of the organs with a cytoplasmic or nuclear genetic factor conferring male sterility or a chemical agent.

Essentially all of the morphological and physiological characteristics: A plant having essentially all of the morphological and physiological characteristics of a designated plant has all of the characteristics of the plant that are otherwise present when compared in the same environment, other than an occasional variant trait that might arise during backcrossing or direct introduction of a transgene.

F₁ Hybrid: The first generation progeny of the cross of two nonisogenic plants.

Fallout (Fo): As used herein, the term “fallout” refers to the rating of how much cotton has fallen on the ground at harvest.

2.5% Fiber Span Length: Refers to the longest 2.5% of a bundle of fibers expressed in inches as measured by High Volume Instrumentation (HVI).

Fiber Characteristics: Refers to fiber qualities such as strength, fiber length, micronaire, fiber elongation, uniformity of fiber and amount of fiber as measured by High Volume Instrumentation (HVI).

Fiber Elongation: Sometimes referred to as E1, refers to the elongation of the fiber at the point of breakage in the strength determination as measured by High Volume Instrumentation (HVI).

Fiber Span Length: The distance spanned by a specific percentage of fibers in a test specimen, where the initial starting point of the scanning in the test is considered 100 percent as measured by High Volume Instrumentation (HVI).

Fiber Strength: Also referred to as T1, denotes the force required to break a bundle of fibers. Fiber strength is expressed in grams per tex on a High Volume Instrument (HVI).

Fruiting Nodes: The number of nodes on the main stem from which arise branches that bear fruit or boll in the first position.

Genotype: The genetic constitution of a cell or organism.

Gin Turnout: Refers to fraction of lint in a machine harvested sample of seed cotton (lint, seed, and trash).

Haploid: A cell or organism having one set of the two sets of chromosomes in a diploid.

Linkage: A phenomenon wherein alleles on the same chromosome tend to segregate together more often than expected by chance if their transmission was independent.

Lint Index: The weight of lint per seed in milligrams.

Lint Percent: Refers to the lint (fiber) fraction of seed cotton (lint and seed).

Lint Yield: Refers to the measure of the quantity of fiber produced on a given unit of land. Presented herein in pounds of lint per acre.

Lint/boll: As used herein, the term “lint/boll” is the weight of lint per boll.

Maturity Rating: A visual rating near harvest on the amount of open bolls on the plant. The rating range is from 1 to 5, 1 being early and 5 being late.

Micronaire: A measure of the fineness of the fiber. Within a cotton cultivar, micronaire is also a measure of maturity. Micronaire differences are governed by changes in perimeter or in cell wall thickness, or by changes in both. Within a variety, cotton perimeter is fairly consistent and maturity will cause a change in micronaire. Consequently, micronaire has a high correlation with maturity within a variety of cotton. Maturity is the degree of development of cell wall thickness. Micronaire may not have a good correlation with maturity between varieties of cotton having different fiber perimeter. Micronaire values range from about 2.0 to 4.3+. Pima micronaire as shown below in Table 2 as defined by USDA classing office:

TABLE 2 Below 2.9 Very fine Possible small perimeter but mature (good fiber), or large perimeter but immature (bad fiber). 2.9 to 3.7 Fine Various degrees of maturity and/or perimeter. 3.8 to 4.2 Average Average degree of maturity and/or perimeter. 4.3 and up Coarse Usually fully developed (mature) but larger perimeter (undesirable fiber).

Pima: The term “Pima” is used in the same context as “American Pima” describes a sub-set of Gossypium barbadense cotton varieties developed in the desert southwest in the early 1900's. In 1908 the USDA started it's G. barbadense breeding program on the Gila River Pima Indian reservation at Sacaton, Ariz. Moved in 1951 to the Cotton Research Center in Phoenix, Ariz. and then moved again in 1984 to Maricopa, Ariz.

Phenotype: The detectable characteristics of a cell or organism, which characteristics are the manifestation of gene expression.

Plant Height: The average height in meters of a group of plants.

Quantitative Trait Loci (QTL): Quantitative trait loci (QTL) refer to genetic loci that control to some degree numerically representable traits that are usually continuously distributed.

Recurrent Parent: The repeating parent (variety) in a backcross breeding program. The recurrent parent is the variety into which a gene or trait is desired to be introduced.

Regeneration: The development of a plant from tissue culture.

Seed/boll: Refers to the number of seeds per boll.

Seedcotton/boll: Refers to the weight of seedcotton per boll.

Seedweight: Refers to the weight of 100 seeds in grams.

Self-pollination: The transfer of pollen from the anther to the stigma of the same plant or a plant of the same genotype.

Single Locus Converted (Conversion) Plant: Plants which are developed by a plant breeding technique called backcrossing wherein essentially all of the morphological and physiological characteristics of a variety are recovered in addition to the characteristics conferred by the single locus transferred into the variety via the backcrossing technique. A single locus may comprise one gene, or in the case of transgenic plants, one or more transgenes integrated into the host genome at a single site (locus).

Stringout Rating: also sometimes referred to as “Storm Resistance” refers to a visual rating prior to harvest of the relative looseness of the seed cotton held in the boll structure on the plant. The rating values are from 1 to 5 (tight to loose in the boll).

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a cotton plant by transformation.

Uniformity Ratio: A measure of the relative fiber span length uniformity of a bundle of fibers. The uniformity ratio is determined by dividing the 50% fiber span length by the 2.5% fiber span length as measured by High Volume Instrumentation (HVI).

Vegetative Nodes: The number of nodes from the cotyledonary node to the first fruiting branch on the main stem of the plant.

As shown in Table 3 below, the foliar damage index and the root vascular stain index for DP 348 RF PIMA is compared with cotton cultivars PHY 805 RF and DP 340.

TABLE 3
Index DP348 RF DP 340 PHY 805RF
Foliar damage index

| | | | |
|---|---|---|---|
| Location 1 | 0.0 | 3.3 | 1.3 |
| Location 2 | 0.0 | 2.0 | 2.0 |

Root vascular strain index

| | | | |
|---|---|---|---|
| Location 1 | 1.1 | 2.5 | 1.3 |
| Location 2 | 0.7 | 1.7 | 1.7. |

As shown in Table 4 below, the Firewall Transgenic identification test of DP 348 RF PIMA for the presence or absence of select genes.

TABLE 4
Transgene trait DP 348 RF PIMA

| | RF | B1 | B2 | R1 | LL | Glycol | DGT | Cry1F | Cry1Ac | Cot102 |
|---|---|---|---|---|---|---|---|---|---|---|
| percent present | 100 | 0.03 | 0.12 | 0 | 0 | 0 | 0 | 0 | 0 | 0. |

4. Deposit Information

A deposit of the cotton variety DP 348 RF PIMA, which is disclosed herein above and referenced in the claims, will be made with the American Type Culture Collection (ATCC), 10801 University Blvd., Manassas, VA 20110-2209. The date of deposit is ______ and the accession number for those deposited seeds of cotton variety DP 348 RF PIMA is ATCC Accession No. PTA-______. All restrictions upon the deposit have been removed, and the deposit is intended to meet all of the requirements of the Budapest Treaty and 37 C.F.R. §1.801-1.809. The deposit will be maintained in the depository for a period of 30 years, or 5 years after the last request, or for the effective life of the patent, whichever is longer, and will be replaced if necessary during that period.

The references cited herein, to the extent that they provide exemplary procedural or other details supplementary to those set forth herein, are specifically incorporated herein by reference.

5. What is claimed is:

1. A seed of cotton variety DP 348 RF PIMA, wherein a representative seed of said variety was deposited as ATCC Accession No ______.

2. A plant part of the plant of claim 1, wherein the plant part comprises at least one cell of said plant.

3. The plant part of claim 2, further defined as pollen, a meristem, a cell, or an ovule.

4. A seed of cotton variety DP 348 RF PIMA, wherein a sample of seed of said variety has been deposited under ATCC Accession No. ______.

5. A cotton plant that expresses all of the morphological and physiological characteristics of cotton variety DP 348 RF PIMA, wherein a sample of seed of said variety has been deposited under ATCC Accession No. ______.

6. A method of producing cotton seed, wherein the method comprises crossing the plant of claim 1 with itself or a second cotton plant.

7. The method of claim 6, wherein the method comprises crossing the plant of cotton variety DP 348 RF PIMA with a second, distinct cotton plant to produce an F₁ hybrid cotton seed.

8. A cotton seed produced by the method of claim 7.

9. A cotton plant produced by growing the seed of claim 8.

10. A composition comprising a seed of cotton variety DP 348 RF PIMA comprised in plant seed growth media, wherein a sample of seed of said variety has been deposited under ATCC Accession No. ______.

11. The composition of claim 10, wherein the growth media is soil or a synthetic cultivation medium.

12. A plant of cotton variety DP 348 RF PIMA further comprising a single locus conversion, wherein a sample of seed of cotton variety DP 348 RF PIMA has been deposited under ATCC Accession No. ______.

13. The plant of claim 12, wherein the single locus conversion comprises a transgene.

14. A seed that produces the plant of claim 12.

15. The seed of claim 14. wherein the single locus confers a trait selected from the group consisting of male sterility, herbicide tolerance, insect or pest resistance, disease resistance. modified fatty acid metabolism, abiotic stress resistance, site-specific genetic recombination, modified carbohydrate metabolism and modified cotton fiber characteristics.

16. The seed of claim 15, wherein the single locus confers tolerance to an herbicide selected from the group consisting of glyphosate, sulfonylurea, imidazolinone, dicamba, glufosinate, phenoxy propionic acid, cyclohexanedione, triazine, benzonitrile, and bromoxynil.

17. The seed of claim 15, wherein the single locus comprises a transgene.

18. The method of claim 7, wherein the method further comprises:
(a) crossing a plant grown from said F₁ hybrid cotton seed with itself or a different cotton plant to produce a seed of a progeny plant of a subsequent generation;
(b) growing a progeny plant of a subsequent generation from said seed of a progeny plant of a subsequent generation and crossing the progeny plant of a subsequent generation with itself or a second plant to produce a progeny plant of a further subsequent generation; and
(c) repeating steps (a) and (b) using said progeny plant of a further subsequent generation from step (b) in place of the plant grown from said F₁ hybrid cotton seed in step (a), wherein steps (a) and (b) are repeated with sufficient inbreeding to produce an inbred cotton plant derived from the cotton variety DP 348 RF PIMA.

19. The method of claim 18, further comprising crossing said inbred cotton plant derived from the cotton variety DP 348 RF PIMA with a plant of a different genotype to produce a seed of a hybrid cotton plant derived from the cotton variety DP 348 RF PIMA.

20. A method of producing a commodity plant product comprising collecting the commodity plant product from a plant of cotton variety DP 348 RF PIMA, wherein a sample of seed of said variety has been deposited under ATCC Accession No. ______.

21. The method of claim 20, wherein the commodity plant product is lint. seed oil. or seed.
